(19) United States
(12) Patent Application Publication
(10) Pub. No.: US 2006/0154884 A1
(43) Pub. Date: Jul. 13, 2006

(54) Use of a vegf receptor gener or gene product

(76) Inventor: Arnd Buchwald, Kiel (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/560,431

(22) PCT Filed: Jun. 18, 2003
(86) PCT No.: PCT/DE03/02048

§ 371 Date: Dec. 14, 2005

(51) Int. Cl. A61K 48/00; A61M 31/00

(52) U.S. Cl. 514/44; 604/500

(57) ABSTRACT

The invention relates to the use of a VEGF receptor gene or receptor for the prevention or treatment of restenoses, ischaemia, arteriosclerosis and other cases of exuberant proliferation. The invention especially relates to the use thereof in therapy following balloon catheter treatment of the coronary vessels. The invention also relates to devices, such as stents, containing the VEGF receptor gene or gene product.

The present invention relates to a novel use of the VEGF receptor gene or gene product in the prevention or treatment of restenosis, ischemia and arteriosclerosis and, in a general manner, in connection with conditions which are linked to an overshooting proliferation of the blood vessel wall cells.

The invention is furthermore directed towards devices for locally administering VEGF receptor gene or gene product, in particular towards stents which comprise the VEGF receptor gene or the receptor.

### BACKGROUND OF THE INVENTION

Approx. 150 000 balloon catheter-assisted dilatations of constricted coronary blood vessels are carried out annually in Germany. According to available controlled studies, approximately 20% of them must be expected to undergo a reconstriction (what is termed restenosis) which is so severe that symptoms occur once again and a fresh treatment is required (Fishman D L, Leon M B, Baim D S, et al.; A randomized comparison of coronary stent placement and balloon angioplasty in the treatment of coronary artery disease; New Engl J Med 1994, 331: 496-501; Serruys P W, De Jaegere P, Kiemeneji F, et al.; A comparison of balloon expandable-stent implantation with balloon angioplasty in patients with coronary artery disease; New Engl J Med 1994, 331: 489-495). The basal cause of this restenosis is an overshooting proliferation (new formation of tissue) of the inner layer (intima) of the blood vessel, with this proliferation leading to a narrowing of the inner space (lumen) (Karas S P, Gravanis M B, Santoian E C, Robinson K A, Anderberg K A, King S B; 3d. Coronary intimal proliferation after balloon injury and stenting in swine: An animal model of restenosis; J Am Coll Cardiol 1992, 20: 467-474; Hoffmann R, Mintz G S, Dussaillant G R, et al. Patterns and mechanisms of in-stents restenosis: A serial ultrasound study; Circulation 1996, 94: 1247-1254). While this process occurs in principle in all patients following such a treatment, it only reaches a critical extent in approx. 20% of the patients. The reasons why only some of the patients are affected have not been fully clarified, just as the signal pathways which control this proliferative healing reaction are not fully known. Generally, this process has been concluded after a few months (as a rule after 6 months or after 9 months according to a few studies), i.e. if there has been no reconstriction by then, there will not be any after that, either. It has furthermore been observed that the proliferation ends when the blood vessel segment once again has a complete inner lining (endothelium) (Terman BI, Dougher-Vermozen M, Carrion M E, Dimitrow D, Armellino D C, Gospodarowicz D, Böbhlen P.; Identification of the KDR tyrosine kinase as a receptor for vascular endothelial growth factor; Biochem Biophys Res Commun 1992, 187: 1579-1586; Clowes A W, Collazzo R E, Karnovsky M J; A morphologic and permeability study of luminal smooth muscle cells after arterial injury in the rat; Lab Invest 1978, 39:141-150). This cell layer, which is normally present, is almost completely destroyed by the arteriosclerosis disease itself or by a balloon dilatation and has to be formed anew. According to the few data available from patients who died shortly after a balloon dilatation and were examined in the context of an autopsy, but especially according to experimental investigations carried out on animals, this regeneration of the endothelium takes several weeks. However, as long as it is going on, the proliferation of the blood vessel wall in this region continues and may potentially lead to a critical reconstriction of the blood vessel lumen.

In the middle of the 1990s, it was shown in an experimental animal model that this endothelial regeneration can be accelerated and that there is then less blood vessel wall proliferation as well (Asahara T, Bauters C, Pastore C, Keamey M, Rossow S, Bunting S, Ferara N, Synes J F, Isner J M; Local delivery of vascular endothelial growth factor accelerates reendothelialization and attenuates intimal hyperplasia in balloon-injured rat carotid artery; Circulation 1995, 91: 2793-2801; Asahara T, Chen D, Tsunumi Y, Kearey M, Rossow S, Passeri J, Symes J F, Isner J M; Accelerated restitution of endothelial integrity and endothelium-dependent function after phVEGF165 gene transfer; Circulation 1996, 94: 3291-3302). For this purpose, the authors used the endothelial cell-specific growth factor VEGF (vascular endothelial growth factor), with the authors either using the VEGF protein or overexpressing the encoding DNA locally in the blood vessel. VEGF is (patho)physiologically formed locally after a blood vessel wall injury (Chen Y X, Nakashima Y, Tanaka K, Shiraishi S, Nakagawa K, Sueishi K; Immunohistochemical expression of vascular endothelial growth factor/vascular permeability factor in atherosclerotic intimas of human coronary arteries. Arterioscler Thromb Vasc Biol 1999, 19: 131-139).

Both approaches for augmenting this growth factor were effective in regard to the blood vessel wall proliferation.

The endothelial activity of VEGF is mediated by two high-affinity tyrosine kinase receptors, i.e. flt-1 and KDR. The murine homolog of the KDR receptor is flk-1. Both receptors have seven immunoglobulin-like domains, a transmembrane domain and an intracellular tyrosine kinase domain. While KDR/flk-1 only binds to VEGF with high affinity, the flt-1 receptor binds with high affinity to PLGF (placenta growth factor) as well as to VEGF.

However, the above-described activity of VEGF can, in both the cases which are highlighted above, i.e. the local use of the protein or the local overexpression of the VEGF-encoding DNA, also lead to an increase, in the blood, of the circulating concentration of VEGF, which does not occur, or does not occur in detectable concentrations, physiologically.

This finding is of considerable importance insofar as VEGF, by means of its endothelial cell growth-promoting effect, plays an essential role in tissue neoformation in malignant tumors. As a result, such a treatment with VEGF or its DNA, for the purpose of avoiding restenosis, would potentially have an undesirable effect which was to be avoided, namely that of promoting the growth of a previously unrecognized tumor.

For this reason, the invention is based, in particular, on the object of preventing, while avoiding the above-described problems, the development of the restenosis which is associated with an experimental balloon catheter treatment and which is caused by an excessive formation of neointimal cells in the treated region. In addition, there was the object of restricting the development of ischemia, arteriosclerosis and tumors and making a treatment possible.

### SUMMARY OF THE INVENTION

In one aspect, the present invention relates to the use of a VEGF receptor gene or gene product (receptor) for preventing or treating restenosis, in particular restenosis which is caused by a balloon catheter treatment of the coronary blood vessels, ischemia or arteriosclerosis.

The invention also specifically relates to the use of a VEGF receptor gene or gene product for producing a preparation which is suitable, in particular, for local administration and can be used for preventing and treating conditions and diseases, in particular arteriosclerosis and ischemia, which are accompanied by overshooting neointimal proliferation, for promoting neovascularizations and for supportive therapy in connection with shunts, for the local treatment of areas of damage to the blood vessel endothelium, in particular before, during or after angioplasty, and for restenosis prophylaxis.

The VEGF receptor gene is, in particular, a sequence which encodes human KDR/flk-1; the gene product is preferably KDR (kinase insert domain-containing receptor) flk-1.

The preparation can comprise further pharmaceutically tolerated additives and auxiliary substances and/or further pharmaceutical active compounds.

It can also, for the purpose of supporting the desired effect according to the invention, comprise agents which modulate the synthesis, expression and/or stability of the receptor at the site of action, for example by modulating the synthesis, expression or stability of a VEGF-receptor-encoding mRNA. It is in this way possible to exert an additional influence on the desired increased presence of the receptor at the site of action.

The preparation can be provided for the administration within a device for supplying it or on, at or in an implant, in particular a stent.

Methods for treating patients who have been subjected to a balloon catheter treatment or for prophylactically treating patients in whom there is a risk of restenosis, ischemia or arteriosclerosis with a VEGF receptor gene or gene product likewise come within the scope of the patent.

The use and the treatment are effected, in particular, by means of the local administration of the VEGF receptor gene or gene product.

An appurtenant method comprises the local administration of an effective quantity of VEGF receptor gene or gene product to the affected regions, with it being possible for the administration to be effected using a stent or a balloon catheter.

Particularly advantageously in this connection, the VEGF receptor is expressed transiently in the affected regions, for example by means of transiently transfecting cells with an expression vector which contains the gene encoding VEGF receptor. In particular, the tissue which has been damaged by a stent treatment or balloon catheter treatment is, according to the invention, transiently transfected with the VEGF receptor gene. This contributes to the regeneration of these tissues and regulates the neoformation of endothelial cells. A single administration will frequently suffice.

The administration can be effected at the time of the balloon catheter-assisted dilatation of constricted cardiac blood vessels.

In another aspect, the invention is directed towards devices, such as a stent or a balloon catheter, which comprise the VEGF receptor gene or gene product.

### BRIEF DESCRIPTION OF THE FIGURES

FIG. 1 shows the detection of the CMV promoter in different tissues of treated animals as a demonstration of the local transfection of cells by the expression vector which has been introduced.

FIG. 2 shows the chronological course of the expression of the VEGF receptor KDR/flk-1 mRNA in transfected animals.

FIG. 3 depicts the area of the lumen and of the neointima in animals which have been treated with the KDR/flk-1 transfection vector and in animals which have been treated with the control.

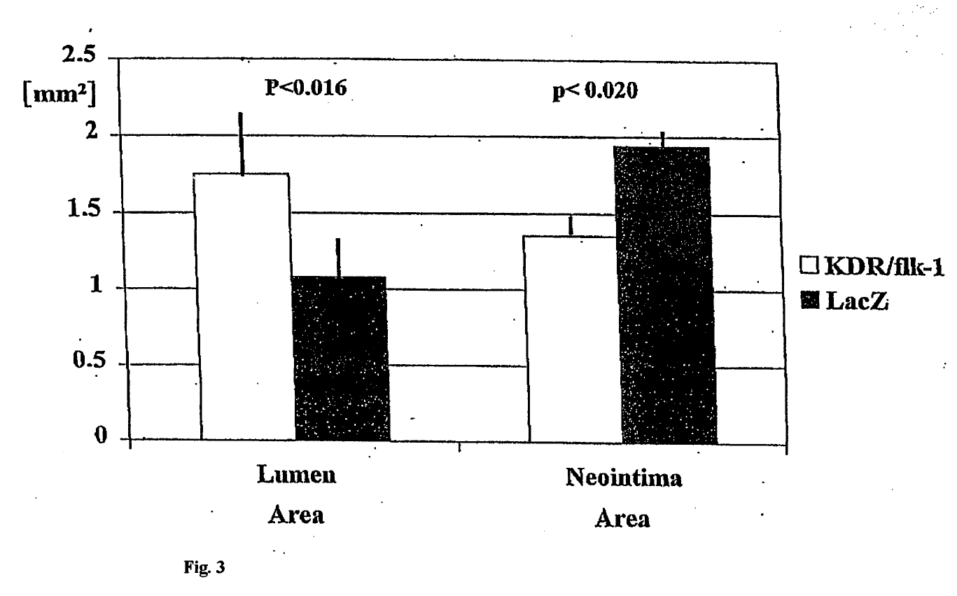

FIG. 4 depicts the thickness of the neointima in animals which have been treated with the KDR/flk-1 transfection vector and in animals which have been treated with the control.

### DETAILED DESCRIPTION OF THE INVENTION

As it is used here, the expression VEGF receptor gene or gene product means any DNA sequences and polypeptides, in particular the KDR/flk-1 receptor (KDR stands for kinase insert domain-containing receptor), its murine homolog flk-1 (flk-1 stands for fetal liver kinase-1) and the DNA sequences and the corresponding degenerate sequences which encode these proteins, which are able, at the protein level, to bind VEGF with high affinity and to elicit the appurtenant signal cascade intracellularly. The receptors which are suitable for the invention also include the tyrosine kinase receptor flt-1. In this connection, both the DNA and the polypeptide can exhibit changes, such as a mutation, e.g. deletion, substitution and/or additional nucleotide or amino acid molecules, in their sequences. These mutations can, for example, comprise from 1 to 20, preferably from 1 to 10, mutations at the protein level. In this connection, it is important that the activity of the VEGF receptor, i.e. to bind VEGF, is preserved. This expression also covers fragments or parts of the VEGF receptor as long as these fragments or parts encompass the VEGF-binding region and are consequently able to bind VEGF.

In one aspect, the present invention relates to the use of a VEGF receptor gene or gene product in the prevention or treatment of restenosis, in particular restenosis which is caused by a balloon catheter treatment of the coronary blood vessels.

The invention furthermore relates, in a second aspect, to the use of a VEGF receptor gene or gene product in the prevention and treatment of ischemia and arteriosclerosis. These diseases can likewise be characterized by expressive proliferation of neointimal cells (blood vessel wall cells).

It is furthermore possible to use the VEGF receptor gene or the receptor to accelerate the lining of what are termed transjugular intrahepatic portosystemic (TIPS) shunts with endothelium. When TIPS stents are used, a channel is created between the portal vein and the vena cava within the liver, with access being gained by way of the jugular vein, in order to lower the elevated portal vein pressure in patients who are suffering from liver cirrhosis and high portal vein pressure. This channel is stabilized with stents. However, a high degree of constriction, resulting from an intense proliferation in the stents, occurs in a substantial proportion of the patients such that consequential treatments become necessary. This proliferative reaction can also be slowed down, and thus a critical constriction prevented, by using the treatment with the VEGF receptor or its DNA to accelerate the formation of endothelium.

Another use according to the invention consists in supporting neovascularizations, by preventing occlusion of the new blood vessels due to overshooting neointimalproliferation, by administering, in particular locally, VEGF receptor or appurtenant DNA. The use according to the invention is suitable in connection with, inter alia, direct myocardial neovascularization. In this procedure, channels measuring 1-2 mm in diameter are created, by means of small bore holes or laser treatment, in segments of the heart muscle in the hope that new blood vessels will be formed from them. This method is employed when conventional methods, such as bypass surgery or balloon dilatation, can no longer be used because the body's own blood vessels are entirely obliterated. Unfortunately, the desired formation of new blood vessels does not usually occur; instead, the channels which have been created close up once again. In this case, therefore, the neoformation of blood vessels can be promoted by treating with VEGF receptor or its DNA.

Methods for treating patients who have undergone a balloon catheter treatment, or for prophylactically treating patients in whom there is a risk of restenosis, ischemia or arteriosclerosis, with a VEGF receptor gene or gene product likewise come within the scope of the patent.

The use and the treatment are effected, in particular, by the VEGF receptor gene or gene product being administered locally.

In this connection, it is particularly advantageous for the VEGF receptor to be expressed transiently in the affected regions, e.g. by means of cells being transfected transiently with an expression vector which contains a DNA sequence which encodes the VEGF receptor or parts thereof.

In another aspect, the invention is directed towards devices, such as a stent, which comprise the VEGF receptor gene or gene product, e.g. in the form of nanoparticles, microparticles, microspheres and nanospheres or as an injectable solution.

The method for treating patients who are, for example, undergoing a balloon catheter treatment comprises the local administration of an adequate quantity of, for example, an expression vector which contains a sequence encoding the VEGF receptor or the protein itself in a manner which, as the end result, makes it possible for a protein which binds VEGF with high affinity, and thus prevents overshooting proliferation of the neointimal cells, to be released in a regulated manner. The appearance of VEGF in the blood of the patient, with its possible disadvantageous consequences, is avoided by the selective, local use of the receptor in place of the factor. The quantity of VEGF receptor gene or gene product to be administered depends on the constitution of the patient, the extent of the treatment, etc., and can readily be determined by the skilled person.

In the case of a balloon catheter treatment, the administration is advantageously effected during this treatment locally at the treatment site. In the case of ischemia, arteriosclerosis, concomitant treatment in connection with shunts or for neovascularization, the VEGF receptor gene or gene product is administered locally in, or in the vicinity of, the treatment site.

The administration can, for example, be effected by the stent being prepared, in connection with the balloon catheter treatment, such that the VEGF receptor gene or gene product is released, for example in the form of an expression vector, into the tissue which is directly adjacent to the stent. The active constituent can consequently, for example, be present in the form of microcapsules, nanocapsules, liposomes or preparations which release in a regulated manner; these latter can be applied to the stent, or parts thereof, in the form of a coating, for example.

On the one hand, this makes it possible, when using expression vectors which contain a VEGF-receptor-encoding DNA sequence, for the surrounding tissue to be transfected, with this resulting in the receptor being expressed. Advantageously, this transfection takes place transiently, e.g. with the target sequence being expressed over a period of from 3 to 4 weeks.

On the other hand, it is also possible to administer recombinant receptor in a form which permits controlled release over a relatively long period of time. This formulation includes nanocapsules and microcapsules and nanospheres and microspheres. This form can, for example, be a recombinant receptor or polypeptide which encompasses the binding domains for VEGF such that it binds VEGF. Where appropriate, the receptor protein can be coupled to a suitable transport vehicle for ensuring accelerated or improved transport into the target cells of the affected tissue or the cell walls of the blood vessel. Transport proteins or transport peptides which are known in the prior art are suitable for this purpose.

The expression vector which can be used for the, where appropriate transient, transfection of the local tissue can be one which is customary employed for use in mammals such as humans.

The administration can also take place in the form of solutions for injection, for example in the case of an ischemia. The VEGF receptor gene or gene product is then formulated together with other appropriate pharmaceutically acceptable components, such as diluents, excipients, etc., and administered to the patient.

The insight which gives rise to the present invention is based, on the one hand, on the finding according to which the agonist VEGF is expressed at an earlier stage, and more strongly, than its receptor during the first days after an experimental balloon catheter treatment (Buchwald A B, Meyer T, Stevens J, et al.; Vascular endothelial growth factor expression in reendothelialisation and neovascularisation in a coronary angioplasty model; 1997, Eur Heart J 18: 154). Consequently, the growth factor VEGF (agonist of the VEGF receptor) is already present at an early point in time while the receptor which is required for mediating the signal is still not being formed. No remote biological effect on already existing (tumor) cells is to be expected when it is not the DNA for the growth factor itself but, instead, that for its receptor, which exerts its effect as a cell-wall protein having 7 transmembrane domains, which is transfected, since there is no incorporation of a protein from the blood into existing cell walls, with subsequent active function, even if local overexpression at a site in the vascular system, such as a coronary artery, were to lead to a measurable circulation of receptor protein in the blood.

It has been shown that local overexpression of the receptor leads to the proliferative blood vessel wall reaction being reduced.

However, this does not only thereby highlight a novel approach for avoiding restenosis following coronary angioplasty but also, at the same time, demonstrates, for the first time, that it is not the presence or the local active concentration of the agonist VEGF which is rate-determining for a biological effect but, instead, that the receptor is in this case crucially important for beginning the effect.

With the aid of the examples, it is shown that local transfection of the DNA for the VEGF receptor KDR/flk-1 using a sideport balloon catheter for the local treatment leads to a marked amplification, by a factor of 10 as compared with control-transfected blood vessels, of the expression of the KDR/flk-1 mRNA. This results in a significant reduction in neointimal proliferation as the essential determinant of in-stent restenosis. By way of example, this effect was achieved by means of a single administration of naked DNA in a CMV promoter at the time of the angioplasty.

These results are the first evidence for it not being only the agonist VEGF, but also its receptor KDR/flk-1, which is rate-determining for the process of the endothelial regeneration which is ultimately proliferation-limiting. According to the invention, it is possible to cause the endogenous expression of VEGF to begin sufficiently rapidly, after an angioplasty, to regenerate the endothelium rapidly and slow down the proliferation in the blood vessel wall when its receptor KDR/flk-1 is available in due time and in adequate quantity.

The extent of the proliferation inhibition which is achieved in accordance with the invention is comparable with that which was found in earlier studies by treating with VEGF. This thereby also provides evidence that this receptor is rate-determining in this model.

The receptor is not overexpressed for a longer period than in control blood vessels or untransfected blood vessels. As to be expected when using naked DNA, for example, stable transfection, leading to long-lasting (over)expression of the receptor, is not achieved. This is also desirable in some applications because, in connection with the balloon catheter treatment, for example, the process of lumen constriction comes to a standstill after the endothelium has regenerated and any further effect would be unnecessary. In other applications, on the other hand, stable transfection can be advantageous.

In addition to this, VEGF which gains access to the blood circulation either when the protein is administered or after local transfection can elicit undesirable effects in the body including the potential danger of the augmentation of blood vessel growth in unrecognized tumors. In accordance with the invention, the transfected DNA was not found to be expressed in any other organ than the target organ. Since, however, the functional KDR/flk-1 receptor has 7 transmembrane domains, it is not to be expected that this protein would have any effect even when it has gained access to the blood circulation. Having an effect would require the receptor to be incorporated from the blood into cell membranes, something which has not thus far been known to occur.

The use, according to the invention, of VEGF receptor gene and gene product can bring about a transfection, which is local where appropriate, of the DNA for the VEGF receptor KDR/flk-1, with the transfection demonstrating a novel approach to the gene therapy of restenosis. It is furthermore also possible, according to the invention, to treat severe arteriosclerotic changes, involving impaired or destroyed endothelium, for example for avoiding plaque rupture with subsequent intravascular thrombosis and cardiac infarction.

It is furthermore possible, according to the invention, to use the VEGF receptor gene and gene product in connection with preventing and treating ischemia.

The device according to the invention, such as the stent comprising the VEGF receptor gene or gene product, can be a conventional stent which is appropriately prepared with the VEGF receptor gene or gene product in a customary formulation.

The invention is explained in more detail below with reference to examples. However, it will be clear to the skilled person that these examples can be altered for the purpose of achieving the present invention. It is rather the case that the examples are used to make the invention more comprehensible.

### EXAMPLES

### Example 1

Minipigs (Relliehausen experimental animal farm) were used as the experimental animal model (Buchwald A B, Unterberg C, Nebehdahl K, Grone H J, Wiegand V; Low-molecular weight heparin reduces neointimal proliferation after coronary stent-implantation in hypercholesterolemic minipigs; Circulation 1992, 86: 531-537; Unterberg C, Sandrock D, Nebendahl K, Buckwald A B; Reduced acute thrombus formation results in decreased neointimal proliferation after coronary angioplasty; J Am Coll Cardiol 1995, 26: 1747-1754). The animals were sedated with azaperone and then anesthetized with halothane, intubated orally and aspirated. The anesthesia was maintained with fentanyl/dipidolor. After a carotid artery had been exposed, a 7 fr guiding catheter was advanced into the ascending aorta under image converter control and the coronary arteries were visualized. A balloon catheter expansion, with stent implantation, was then carried out in 2 vessels. Immediately after that, an Infiltrator catheter was advanced to both treated sites. When the balloon of this catheter was inflated, 2 rows with in each case 5 apertures were pressed against the vessel wall, with the DNA (in each case 0.2 mg, either KDR/flk 1 or LacZDNA, see below) being injected through them in a volume of 0.4 ml. The personnel who were carrying out the experiments did not know which vessel was treated with the KDR/flk-1 DNA. After that, the balloon was let down, the catheters were removed, the neck wound was sutured and the anesthesia was terminated.

Of 22 animals in this investigation, 3 died following irreversible ventricular fibrillation as a consequence of coronary spasms and subsequent myocardial ischemia after the initial angioplasty prior to the DNA injection. These animals were disregarded in the following evaluation. All the other animals survived without complications until the planned end of the experiment.

The animals were kept in their stalls for the duration of the planned follow-up period. This amounted to 2, 4, 7 or 28 days. The hearts were then removed from the animals after the thorax had been opened under deep, irreversible anesthesia. After the hearts had been fixed in phosphate-buffered sodium chloride solution by perfusion at 100 mmHg, they were fixed by perfusion with 4% formaldehyde (1000 ml). The treated blood vessel segments were removed and embedded in methyl methacrylate. Following elastic van Giesson staining, 3-5 sections (0.4 μm) were analyzed morphometrically using a digital microscope camera and the ImagePro™ program (version 2.0, Media Cybernetics, Silver Spring, USA) . The areas of lumen and newly formed intima and the thickness of this neointima over each stent wire cut end were measured. The depth of penetration or the degree of wounding by the stent was determined semiquantitatively, for each section, on a scale of from 1 (superficial) to 4 (wire in the adventitia), as described by Schwartz et al. (Schwartz R S, Huber K C, Murphy J G, et al.; Restenosis and the proportional neointimal response to coronary artery injury: Results in a porcine model; J Am Coll Cardiol 1992, 19: 267-274). The evaluator did not know the nature of the treatment of the segments.

Proliferation of the blood vessel wall following angioplasty

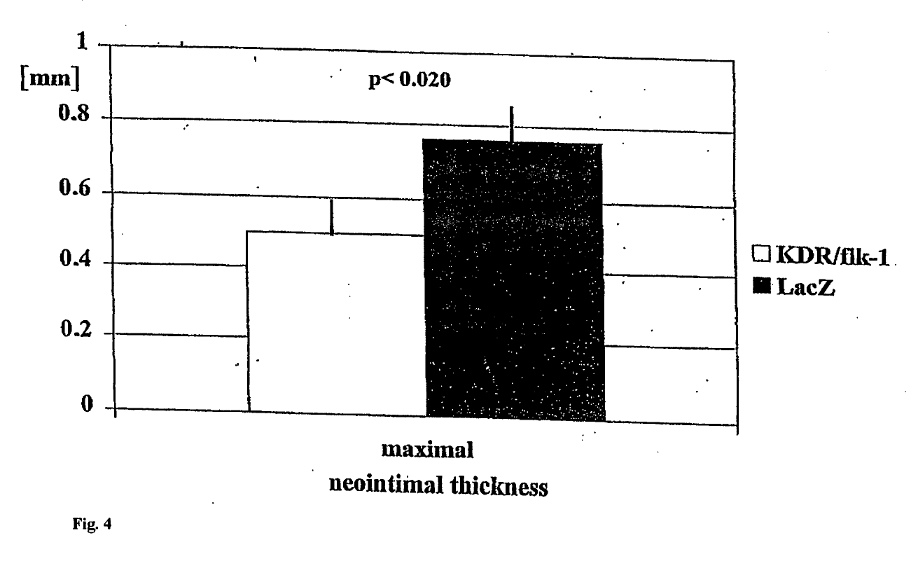

With values of 2.08±0.11 and, respectively, 2.10±0.12, the degree of wounding in KDR/flk-1-transfected experimental animals and in lacZ-transfected controls was comparable. The minimal lumen area was larger, and the neointimal area (FIG. 4), as well as the maximal neointimal thickness (FIG. 4), were smaller, in KDR/flk-1 transfected blood vessels than in LacZ-treated blood vessels. These differences, which constituted an average gain in lumen by half the values in the LacZ-treated blood vessels, or a reduction in the neointimal area by half, were significant.

For the purpose of carrying out the in-situ hybridization for detecting the mRNA, pieces of 3 mm in length were separated off from the blood vessel segments before the embedding in methyl methacrylate; the stent wires were then removed from these pieces, which were embedded in paraffin.

DNA employed: a eukaryotic expression vector which contained the cytomegalovirus promoter pcDNA3.1 (Invitrogen, Groningen, the Netherlands) and the linearized cDNA for human VEGF receptor KDR/flk-1 (Waltenberger J, Claesson-Welsh L, Siegbahn A, Shibuya M, Heldin C H; Different signal transduction properties of KDR and Flt 1, two receptors for vascular endothelial growth factor; Biol Chem 1994, 269: 26 988-26 995) was used. The plasmid pcDNA 3 LacZ (Invitrogen, Groningen, the Netherlands), which contained a “nuclear targeted” β-galactosidase sequence coupled to the promoter, was used for control transfections.

In order to test for successful transfection and to rule out expression of the transfected DNA in other organs, samples from liver, spleen, kidneys and lung were examined for the presence of the CMV promoter mRNA. In order to confirm that transfection had been successful, an in-situ hybridization was carried out using primers for the CMV promoter gene.

Tissue sections were deparaffinized under RNAse-free conditions, fixed with paraformaldehyde, partially digested with protein kinase K (Sigma, Munich), dehydrated once again and then added to a hybridization mix containing a digoxigenin-labeled CMV promoter probe. This probe was prepared using the PCR DG probe synthesis kit (Roche, Mannheim) and the following primers: 5′ GCT GAC CGC CCA ACG AC 3′ and TAC ACG CCT ACC GCC CAT TT 3′; this results in a probe comprising 448 base pairs. An anti-digoxigenin antibody was added stained using the NBT/BCIP staining kit (DAKO, Hamburg).

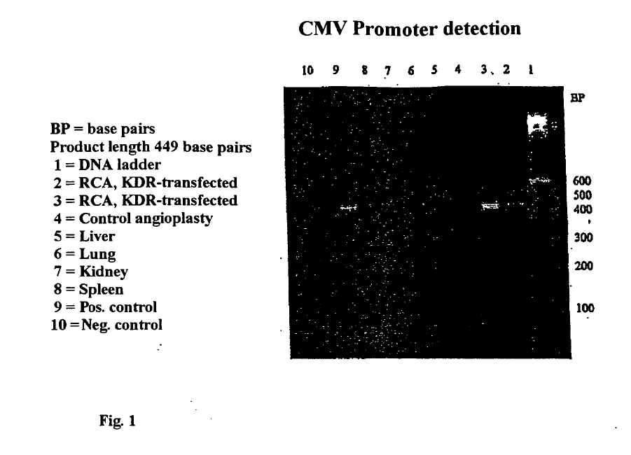

The mRNA was only detected in the transfected blood vessels; all the other organ samples investigated were negative (FIG. 1). Experiments involving follow-up periods of 2 days (n=2), 4 days (n=4) and 7 days (n=3) as well as 4 weeks (n=10) were analyzed for this investigation.

Expression of KDR/flk-1

In order to confirm that transfection had been successful, an in-situ hybridization was carried out using primers for the CMV promoter gene. The CMV promoter gene was selected since in-situ hybridization for KDR/flk-1 is positive in both transfected and control-dilatated animals due to the endogenous expression of this receptor. It was not possible to differentiate between the mRNA which was formed after transfecting the (human) DNA and the endogenous (porcine) mRNA since the complete sequence of the porcine DNA was not known and it was not possible, either, to synthesize specific primers because of the high degree of homology between the two species.

In-Situ Hybridization

Blood vessel sections were deparaffinized under RNAase-free conditions, fixed with paraformaldehyde, partially digested with protein kinase K (Sigma, Munich), dehydrated once again and then added to a hybridization mix containing a digoxigenin-labeled KDR/flk-1 probe. This probe was prepared using the PCR DG probe synthesis kit (Roche, Mannheim) and the following primers: 5′ GAA CTT GGA TAC TCT TTG G 3′ and 5′ CTG CGG ATA GTG AGG TTC 3′; a probe comprising 365 base pairs was obtained. An anti-digoxigenin antibody was added and stained using the NBT/BCIP staining kit (DAKO, Hamburg).

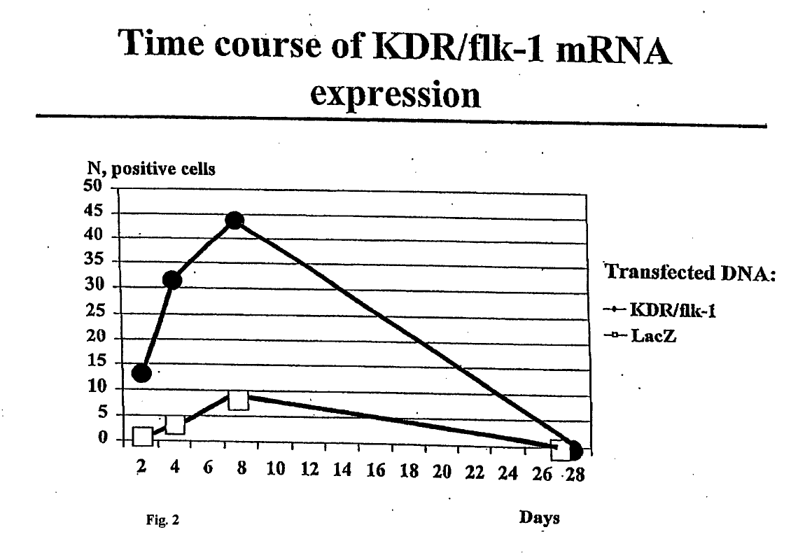

When mRNA expression was analyzed semiquantitatively in the coronary arteries, it was possible to detect the mRNA for KDR/flk-1 by in-situ hybridization in transfected blood vessels after 4 days. The expression was at a maximum after 7 days; after 4 weeks, it was no longer possible to detect any mRNA. By contrast, the magnitude of the positive detection was markedly less in LacZ-transfected blood vessels. FIG. 2 shows a typical finding in transfected blood vessels after 7 days. While staining can be seen, in particular, in periluminal cell layers, this staining is substantially more intensive in KDR/flk-1-transfected blood vessels. FIG. 2 depicts the time course of KDR/flk-1 mRNA expression in both treatment groups.

The depicted results are given as mean values ±standard deviation (SD) or standard error of the mean (SEM) (in each case as appropriate). Neointimal thickness, neointimal area and lumen area following KDR transfection were compared with LacZ controls using the Wilcoxon signed rank test for dependent variables.

1. The use of a VEGF receptor gene or gene product for producing a preparation for preventing and treating conditions and diseases, in particular arteriosclerosis and ischemia, which are accompanied by overshooting neointimal proliferation, for promoting neovascularization and for supportive therapy in connection with shunts, for the local treatment of areas of damage to the blood vessel endothelium, in particular before, during or after angioplasty, and for restenosis prophylaxis.

2. The use as claimed in claim 1, characterized in that the preparation comprises a KDR-encoding sequence as the VEGF receptor gene and/or KDR (kinase insert domain-containing receptor) as the gene product.

3. The use as claimed in claim 1, characterized in that the gene/sequence is present in an expression vector, preferably functionally assigned to a promoter.

4. The use as claimed in claim 1, characterized in that the gene product is assigned to a transport unit, in particular in combination with a transport protein.

5. The use as claimed in claim 1, characterized in that the preparation additionally comprises means for modulating the synthesis, expression and/or stability of a VEGF-receptor-encoding mRNA.

6. The use as claimed in claim 1, characterized in that the preparation comprises further pharmaceutically tolerated additives and auxiliary substances and/or further pharmaceutical active compounds.

7. The use as claimed in claim 1, characterized in that the preparation is provided within a device for supplying it or on, at or in an implant, in particular a stent.

8. The use of a VEGF receptor gene or gene product for preventing and treating conditions and diseases, in particular arteriosclerosis and ischemia, which are accompanied by overshooting neointimal proliferation, for promoting neovascularization and for supportive therapy in connection with shunts, for local treatment of areas of damage to the blood vessel endothelium, in particular before, during or after angioplasty, and for restenosis prophylaxis.

9. The use of a VEGF receptor gene or gene product as claimed in claim 8 for therapy in balloon catheter-assisted dilatation in connection with constricted coronary blood vessels.

10. The use as claimed in claim 8, wherein a VEGF receptor gene is used in an expression vector.

11. The use as claimed in claim 10, wherein transient expression of the VEGF receptor is induced.

12. The use as claimed in claim 1, wherein the VEGF receptor gene or gene product is present in a form in which it is encapsulated by nanoparticles, microparticles or microspheres, in the form of a controlled release system or in the form of a solution.

13. The use as claimed in claim 1, wherein the VEGF receptor gene or gene product is impregnated in or on a stent or stored in an infiltration balloon catheter or a sideport balloon catheter.

14. A device, in particular stent or catheter, for use within blood vessels, comprising an effective quantity of a VEGF receptor gene or a VEGF receptor, or functional moieties thereof having the same biological effect.
